(19) United States
(12) Patent Application Publication
(10) Pub. No.: US 2025/0050039 A1
(43) Pub. Date: Feb. 13, 2025

(54) POWDER INHALER

(71) Applicant: CHIESI FARMACEUTICI S.P.A., Parma (IT)

(72) Inventors: Marco DI CASTRI, PARMA (IT); Giuseppe Antonio MULAS, PARMA (IT); Alan TWEEDIE, PARMA (IT); Ralph Donald Quentin COLLINGS, PARMA (IT); Daniel David HIGGINS, PARMA (IT); Benjamin Michael RICHARDS, PARMA (IT); Odysseas Michail VARVOUNIS, PARMA (IT)

(21) Appl. No.: 18/721,184

(22) PCT Filed: Dec. 19, 2022
(86) PCT No.: PCT/EP2022/086687

§ 371 Date: Jun. 17, 2024

(30) Foreign Application Priority Data
Dec. 21, 2021 (EP) 21216553.4

(51) Int. Cl. A61M 11/02; A61M 15/00

(52) U.S. Cl. CPC A61M 11/02; A61M 15/0071; A61M 2202/062; A61M 2202/064

(57) ABSTRACT

A powder inhaler includes a casing having a mouthpiece, an inhalation channel, a container for a powdered medicament, and a metering device with a shuttle having a dosing recess. The shuttle is movable between a filling position, in which the dosing recess is in alignment with an opening of the container to be filled with a dose of the powdered medicament, and an inhalation position, in which the dosing recess is in alignment with the inhalation channel, for enabling inhalation of the dose of the powdered medicament contained in the dosing recess through the mouthpiece. A sealing device is operationally active at a coupling zone of the shuttle with the container when the shuttle is in the filling position, the coupling zone circumscribing the dosing recess and the opening.

### TECHNICAL FIELD OF THE INVENTION

The present invention relates to a dry powder inhaler, i.e. a device for dispensing a powdered medicament preparation by inhalation. The device is in particular a portable, multiple-dose, breath activated dry powder inhaler without propellant gas, equipped with a metering device which dispenses doses from a medicament container.

### BACKGROUND ART

Inhalers are hand-held portable devices that deliver medication directly to the lungs. One class of inhalers is passive dry powder inhalers (“DPI”). A passive DPI is a patient driven device wherein the action of breathing in through the device draws the powder formulation of a medicament into the respiratory tract. DPIs are well recognized as devices for drug delivery to the lungs for treatment of pulmonary and systemic diseases. They can generally be divided in: i) single-dose (unit-dose) inhalers, for the administration of an individual dose of the active ingredient/s contained in capsule or blister loaded into the device and punctured by the patient immediately before use; ii) pre-metered multi-dose inhalers containing a series of blisters or capsules with the active ingredient/s formulation or iii) reservoir inhalers containing a larger amount of the powder formulation of active ingredient/s, corresponding to multiple doses, which is metered from a storage unit just before inhalation.

Document WO 2004/012801, by the same Applicant, discloses a powder inhaler comprising a casing with a lower shell and an integral cover pivotably coupled to the lower shell. The lower shell delimits a mouthpiece and the integral cover is movable between a closed position in which the mouthpiece is enclosed and hidden by the integral cover and an open position in which the mouthpiece is exposed for use. The lower shell houses a container for storing a powdered medicament, a metering member having a dosing recess to be filled with a dose of the powdered medicament and an inhalation channel in communication with the mouthpiece. The metering member is moveable between a filling position, in which the dosing recess is in alignment with an opening of the container so as to be filled with a dose of the powdered medicament, and an inhalation position, in which the dosing recess is in alignment with an inhalation channel for enabling inhalation of the dose of the powdered medicament contained in the dosing recess. The powder inhaler further comprises a protective member which is slidingly moveable on the metering member between a closed position, in which the protective member covers the dosing recess of the metering member if the metering member is in an inhalation position, and an open position, in which the protective member exposes the dosing recess thereby enabling inhalation of the dose of the powdered medicament contained in the dosing recess. The protective member is coupled to an inhalation actuated mechanism in such a manner that the inhalation actuated mechanism moves the protective member from its closed position to its open position if there is an inhalation suction force exerted by a user which exceeds a predetermined level.

Document WO 2016/000983, by the same Applicant, discloses a powder inhaler similar to the one of WO 2004/012801.

A formulation for powder inhalers is commonly a powder blend of one or more active ingredients dispersed in a pharmacologically inert bulk solid, comprising a physiologically acceptable diluent, such as lactose, and optional additional excipients such as lubricants. The particle size of inhalable active ingredients should be optimized to deliver the drug deep into the lung to achieve efficacy. This efficacious particle size typically lies between 1-6 μm whereas larger particles, in the range 6-10 μm, tend to be deposited in the upper airways without reaching the site of action in the lower airways.

It is well known that stability of the powder as well as the aerosol performances could be affected by environmental conditions, humidity in particular. Therefore, it is desirable to control the humidity within powder inhalers and in particular in reservoir DPIs. There is the need of more efficacious systems to protect from humidity, in particular but not solely, when the drug-containing powder formulation from reservoir DPIs are intended for being stored and used in sub-tropical and tropical countries.

Powder inhalers provided with sealing devices are known.

For instance, document EP2063940B1 discloses an inhalation device for powder drugs comprising a storage chamber for accommodating a plurality of drug powder doses and a dosing device which includes at least one dosing slider which is movable with a translatory movement in a dosing slider passage from a filling position into an emptying position. The dosing slider passage has open end with an opening through which the dosing slider can issue with the dosing cavity, wherein a contact surface for a seal is provided around the opening and wherein the dosing slider further has a sealing surface which is arranged in a plane in approximately transverse relationship with the direction of movement out of the filling position into the emptying position. The seal may comprise a thermoplastic elastomer or a sealing rib which may be formed on the dosing slider passage or in the dosing slider.

Document EP1231964B1 discloses a powder inhaler comprising a powder container, an air channel, a metering member equipped with a dosing recess, an actuating means for the displacement of the metering member between the filling and the inhalation position, and a closure element for plugging the air channel in a substantially water-proof manner when the metering member is in the filling position and opening the air channel when the metering member is in the inhalation position.

### SUMMARY

It is an object of the present invention to provide a powder inhaler with a further improved protection from environmental conditions, like humidity, temperature, light, pressure.

In particular, it is an object of the present invention to provide a powder inhaler which is able to control the humidity inside the inhaler itself.

It is object of the present invention to prevent or at least to limit the entry of atmospheric moisture inside the inhaler and in particular inside the container storing the powdered medicament during distribution and storage at a pharmacy (i.e. before delivering the inhaler to the user) and also after delivering the inhaler to the user, during the time period (e.g. days, months) when the user makes use of the inhaler.

It is also object of the present invention to prevent dispersion the powdered medicament contained in the container.

At least one of the above objects is substantially achieved by a powder inhaler according to one or more of the appended claims and/or of the following aspects.

In accordance with a 1^{st} independent aspect, a powder inhaler, comprises:
a casing having a mouthpiece;
an inhalation channel housed in the casing and connected to the mouthpiece;
a container housed in the casing for storing a powdered medicament;
a metering device comprising a shuttle having a dosing recess, wherein the shuttle is movable between a filling position, in which the dosing recess is in alignment with an opening of the container and faces said opening so as to be filled with a dose of the powdered medicament, and an inhalation position, in which the dosing recess is in alignment with the inhalation channel, for enabling inhalation of the dose of the powdered medicament contained in the dosing recess through the mouthpiece;
wherein the powder inhaler further comprises a sealing device operationally active at a coupling zone of the shuttle with the container when the shuttle is in the filling position, said coupling zone circumscribing the dosing recess and the opening.

The invention allows to improve the stability of the powder and the aerosol performances (i.e. the capability of delivering the drug deep into the lung to achieve efficacy) by protecting said powder from the environmental conditions, particularly from humidity.

In particular, the invention allows to prevent or at least reduce moisture entry through gaps between the shuttle and the container when the shuttle is in the filling position.

Therefore, the invention allows to protect the powder during the period when the inhaler is in use but is closed and stored in a bag, in a pocket, in the open air, in a drawer or in a furniture or other place between inhalations.

The invention allows also to prevent or at least reduce powdered medicament losses through gaps between the shuttle and the container.

Other aspects of the invention are disclosed in the following.

In a 2^{nd} aspect according to aspect 1, the shuttle comprises a main part and a dosing part; wherein the dosing part comprises the dosing recess.

In a 3^{rd} aspect according to aspect 2, the sealing device comprises a deformable portion surrounding the dosing recess and connecting the dosing part to the main part.

In a 4^{th} aspect according to aspect 3, the deformable portion is less stiff than the main part along a direction perpendicular to a lying plane of the shuttle.

In a 5^{th} aspect according to aspect 3 or 4, at least one spring, optionally a plurality of springs, is interposed between the casing and the dosing part and is configured to push said dosing part against the opening of the container by deforming the deformable portion when the shuttle is in the filling position.

The word “less stiff” referred to the deformable portion in this description and claims means that, when the spring pushes the dosing part, the deformable portion deforms more than the dosing part and the main part, so as to allow the dosing part to slightly move towards the opening and to join to the container at the coupling zone while the main part remains in its place. In other words, the deformable portion is more flexible than the dosing part and the main part, so that the connection between the dosing part and the main part does not prevent the dosing part from adapting its position to the shape and position of the container and allows a zone surrounding the dosing recess to adhere to an edge of the opening.

In a 6^{th} aspect according to any of the preceding aspects 1 to 5, the dosing recess is cup shaped.

In a 7^{th} aspect according to any of the preceding aspects 1 to 6, the dosing recess has a circular edge.

In a 8^{th} aspect according to aspect 3 or to any of aspects 4 to 7 when according to aspect 3, the deformable portion is flat.

In a 9^{th} aspect according to aspect 3 or to any of aspects 4 to 8 when according to aspect 3, the deformable portion is shaped like a band surrounding the dosing recess.

In a 10^{th} aspect according to aspect 3 or to any of aspects 4 to 9 when according to aspect 3, the shuttle is shaped like a plate and the main part has a first wall thickness (t1), wherein the deformable portion has a second wall thickness (t2) equal to or smaller than the first wall thickness (t1).

In a 11^{th} aspect according to aspect 10, a ratio t2/t1 of the second wall thickness (t2) to the first wall thickness (t1) is smaller than 0.9, optionally smaller than 0.5; optionally said ratio t2/t1 of the second wall thickness (t2) to the first wall thickness (t1) is greater than 0.1, optionally greater than 0.4.

In a 12^{th} aspect according to aspect 3 or to any of aspects 4 to 11 when according to aspect 3, the deformable portion extends radially with respect to a central axis of the dosing recess perpendicular to the shuttle and has an average width (wₐᵥ) measured along radial directions.

In a 13^{th} aspect according to the preceding aspect 10, 11 or 12, the deformable portion has an average width (wₐᵥ) and a ratio t2/wₐᵥ of the second wall thickness (t2) to the average width (wₐᵥ) is smaller than 0.4, optionally smaller than 0.3; optionally said ratio t2/wₐᵥ of the second wall thickness (t2) to the average width (wₐᵥ) is greater than 0.15, optionally greater than 0.2.

In a 14^{th} aspect according to aspect 3 or to any of aspects 4 to 13 when according to aspect 3, the dosing part comprises a peripheral stiffening rib surrounded by the deformable portion.

In a 15^{th} aspect according to the preceding aspect 14, the peripheral stiffening rib is fashioned on a side of the shuttle opposite the dosing recess.

In a 16^{th} aspect according to aspect 3 or to any of aspects 4 to 15 when according to aspect 3, the main part, the deformable portion and the dosing part are made in a single piece.

In a 17^{th} aspect according to the preceding aspect 16, the single piece is made of plastic.

In a 18^{th} aspect according to the preceding aspect 17, the single piece is made of at least one rigid thermoplastic material, optionally of the following materials: polycarbonate (PC) or polypropylene (PP), acrylonitrile-butadiene-styrene copolymer (ABS), cyclic olefin copolymer (COC).

In a 19^{th} aspect according to aspect 10 or to any of aspects 11 to 18 when according to aspect 10, the first wall thickness (t1) of the main part is between 0.8 mm and 1.85 mm, optionally between 0.9 mm and 1.5 mm, optionally of 1.1 mm.

In a 20^{th} aspect according to aspect 10 or to any of aspects 11 to 19 when according to aspect 10, the second wall thickness (t2) of the deformable portion is the same or substantially the same as the first wall thickness (t1); otherwise, the second wall thickness (t2) of the deformable portion is between 0.3 mm and 0.7 mm, optionally between 0.4 and 0.55 mm, optionally of 0.45 mm.

In a 21^{st} aspect according to aspect 12 or to any of aspects 13 to 20 when according to aspect 12, the average width (wₐᵥ) of the deformable portion is between 1.3 mm and 2.5 mm, optionally of 1.9 mm.

In a 22^{nd} aspect according to aspect 3 or to any of aspects 4 to 21 when according to aspect 3, the deformable portion is made of or comprises an elastomeric material.

In a 23^{rd} aspect according to the preceding aspects 22, the deformable portion is made of at least one of the following materials: silicone, thermoplastic elastomer (TPE), in particular TPE for medical—pharmaceutical applications, for instance styrene block copolymers (TPS (TPE-s)), thermoplastic polyolefin elastomers (TPO (TPE-o)), thermoplastic vulcanizates (TPV (TPE-v or TPV)), thermoplastic polyurethanes (TPU), thermoplastic copolyesters (TPC (TPE-E)), thermoplastic polyamides (TPA (TPE-A)), not classified thermoplastic elastomers (TPZ).

In a 24^{th} aspect according to aspect 3 or to any of aspects 4 to 23 when according to aspect 3, the deformable portion is co-molded with or over-molded to the main part and the dosing part.

In a 25^{th} aspect according to aspect 2 or to any of aspects 3 to 24 when according to aspect 2, the dosing part comprises at least one stiffening rib on a side of the shuttle, in particular of the dosing part, opposite the dosing recess.

In a 26^{th} aspect according to the preceding aspect 25, the at least one stiffening rib is straight and passes through a central axis of the dosing recess.

In a 27^{th} aspect according to the preceding aspect 25 or 26, the at least one stiffening rib is not connected to the main part.

In a 28^{th} aspect according to any of the preceding aspects 25 to 27, the at least one stiffening rib is aligned to a direction of movement of the shuttle between the filling position and the inhalation position.

In a 29^{th} aspect according to aspect 2 or to any of aspects 3 to 28 when according to aspect 2, the shuttle comprises at least one connecting rib, optionally a plurality of connecting ribs, joining the main part to the dosing part.

In a 30^{th} aspect according to the preceding aspect 29, the at least one connecting rib is aligned to a direction of movement of the shuttle between the filling position and the inhalation position.

In a 31^{st} aspect according to aspect 5 or to any of the preceding aspects 6 to 30 when according to aspect 5, the at least one spring contacts the dosing part at least at a center of the dosing recess.

In a 32^{nd} aspect according to aspect 5 or to any of the preceding aspects 6 to 31 when according to aspect 5, the at least one spring contacts the dosing part on the peripheral stiffening rib.

In a 33^{rd} aspect according to aspect 1, the sealing device comprises a gasket placed at the coupling zone, at least when the shuttle is in the filling position.

In a 34^{th} aspect according to aspect 33, the gasket is placed around the opening of the container and, when the shuttle is in the filling position, the gasket is placed around the dosing recess.

In a 35^{th} aspect according to aspect 33 or 34, the inhaler comprises a support plate sandwiched between the container and the shuttle and having a through opening facing the opening of the container, wherein the gasket is placed around said through opening.

In a 36^{th} aspect according to aspect 35, the gasket is placed on an edge of said through opening.

In a 37^{th} aspect according to any of the preceding aspects 33 to 36, the gasket is made of an elastomeric material.

In a 38^{th} aspect according to any of the preceding aspects 33 to 37, the gasket is made at least one of the following materials: silicone, thermoplastic elastomer (TPE), in particular TPE for medical-pharmaceutical applications, for instance styrene block copolymers (TPS (TPE-s)), thermoplastic polyolefin elastomers (TPO (TPE-o)), thermoplastic vulcanizates (TPV (TPE-v or TPV)), thermoplastic polyurethanes (TPU), thermoplastic copolyesters (TPC (TPE-E)), thermoplastic polyamides (TPA (TPE-A)), not classified thermoplastic elastomers (TPZ).

In a 39^{th} aspect according to aspect 35 or to any of aspects 36 to 38 when according to aspect 35, the gasket is co-molded with or over-molded to the support plate.

In a 40^{th} aspect according to aspect 35 or to any of aspects 36 to 39 when according to aspect 35, the support plate is made of plastic.

In a 41^{st} aspect according to aspect 35 or to any of aspects 36 to 40 when according to aspect 35, the support plate is made of polycarbonate (PC) or polypropylene (PP), acrylonitrile-butadiene-styrene copolymer (ABS), cyclic olefin copolymer (COC).

In a 42^{nd} aspect according to any of the preceding aspects 33 to 41, the gasket has a raised bead protruding towards the container.

In a 43^{rd} aspect according to any of the preceding aspects 33 to 42, the gasket has a raised bead protruding towards the shuttle.

In a 44^{th} aspect according to aspect 35 or to any of aspects 36 to 43 when according to aspect 35, the support plate is mounted fixed relative to the casing.

In a 45^{th} aspect according to aspect 35 or to any of aspects 36 to 44 when according to aspect 35, the support plate extends substantially over an entire bottom wall of the casing and has a through inhalation passage in alignment with the inhalation channel, to allow flow of the dose of the powdered medicament contained in the dosing recess when the shuttle is in the inhalation position.

In a 46^{th} aspect according to the preceding aspect 45, a de-agglomerator arrangement is coupled to an end of the inhalation channel opposite the mouthpiece; the de-agglomerator arrangement comprising a vortex chamber and the through inhalation passage copying a shape of said vortex chamber.

In a 47^{th} aspect according to any of the preceding aspects, the powder inhaler comprises a desiccant chamber placed inside the casing and containing or configured to contain a desiccant, wherein the desiccant comprises molecular sieves.

In a 48^{th} aspect according to the preceding aspect 47, the desiccant is contained in a bag able of being inserted in the desiccant chamber or the desiccant is in the form of a single tablet able of being inserted in the desiccant chamber.

In a 49^{th} aspect according to the preceding aspect 48 or 49, the desiccant chamber is part of the container for the powdered medicament.

In a 50^{th} aspect according to any of the preceding aspects 47 to 49, a permeable membrane separates the desiccant chamber from a medicament chamber delimited by the container; the medicament chamber being configured to contain or containing the powdered medicament.

In a 51^{st} aspect according to the preceding aspect 50, a permeability of the permeable membrane is different from, in particular greater than, that between either the desiccant or the medicament and the outside environment.

In a 52^{nd} aspect according to any of the preceding aspect 47 to 51, the molecular sieves are made of a material with pores of uniform size configured to absorb small molecules, such as molecules of water.

In a 53^{rd} aspect according to the preceding aspect 52, the molecular sieves comprise alkaline salts of aluminosilicates, called zeolites, or aluminophosphates or porous glass or active carbon or artificial zeolites.

In a 54^{th} aspect according to any of the preceding aspects 47 to 53, the powder inhaler further comprises the powdered medicament and/or the desiccant.

In a 55^{th} aspect according to any of the preceding aspects, the powdered medicament is a pharmaceutical composition comprising a pharmaceutically acceptable salt of formoterol, optionally in combination with a pharmaceutically acceptable salt of glycopyrronium and/or beclometasone dipropionate (BDP).

In a 56^{th} aspect according to any of the previous aspects, the powder inhaler comprises a cover engageable with the casing to close the mouthpiece; the cover comprising a sealing element, the sealing element comprising a material (optionally silicon or a thermoplastic elastomer (TPE)) that is more deformable than a material of the mouthpiece; when the cover is engaged with the casing and closes the mouthpiece, a first portion of the sealing element is coupled to an opening of the mouthpiece to tight close said opening of the mouthpiece.

In a 57^{th} aspect according to the preceding aspect 56, when the cover is engaged with the casing and closes the mouthpiece, a second portion of the sealing element is coupled to the at least one air inlet to tight close said at least one air inlet.

In a 58^{th} aspect according to any of the preceding aspects, the powder inhaler comprises a dose counting unit contained into the casing and coupled to an inhalation actuated mechanism and/or to the cover; optionally, the casing comprises a window or an opening for displaying the number of doses taken or the number of doses left in the powder inhaler, said number being counted by the dose counting unit.

### DESCRIPTION OF THE DRAWINGS

FIG. 1 shows a 3D view of a powder inhaler according to the present invention in an open configuration;

FIGS. 2A, 2B and 2C are section views of the powder inhaler of FIG. 1 in different states;

FIGS. 3 and 4 show 3D views of one element of the powder inhaler of FIG. 1;

FIG. 5 is a plan view of one side of the element of FIG. 3;

FIG. 6 is a side section view of the element of FIG. 3;

FIG. 7 is 3D view of another element of the powder inhaler of FIG. 1;

FIG. 8 is a plan view of the element of FIG. 7;

FIG. 9 is a section view of a powder inhaler according to the present invention provided with a variant of the element of FIGS. 3 to 6;

FIGS. 10, 11 and 12 show 3D views of the variant element of FIG. 9;

FIG. 13 is a side section view of the element of FIGS. 10, 11, 12;

FIG. 14 is a section view of a different embodiment of a powder inhaler according to the present invention;

FIG. 15 is an exploded view of some components of the inhaler of FIG. 14;

FIG. 16 shows a 3D view of one element of the powder inhaler of FIGS. 14 and 15;

FIG. 17 is an enlarged section view of a portion of the element of FIG. 16;

FIG. 18 is a chart showing a differential weight per day, with respect to a standard powder inhaler, due to moisture absorption of the different embodiments/variants of the invention;

FIG. 19 is a chart showing a cumulative weight over time due to moisture absorption in a standard powder inhaler compared with the embodiments/variants of the invention.

### DETAILED DESCRIPTION

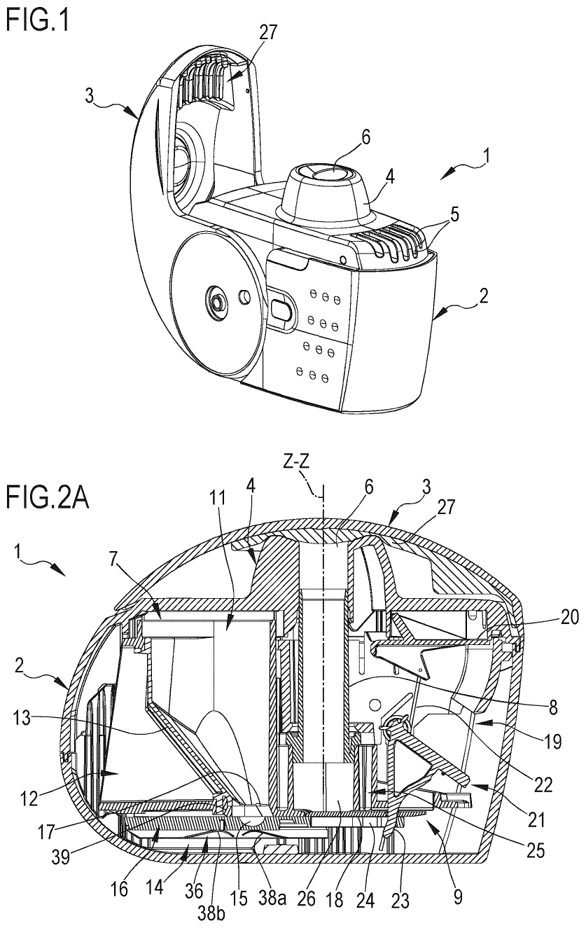

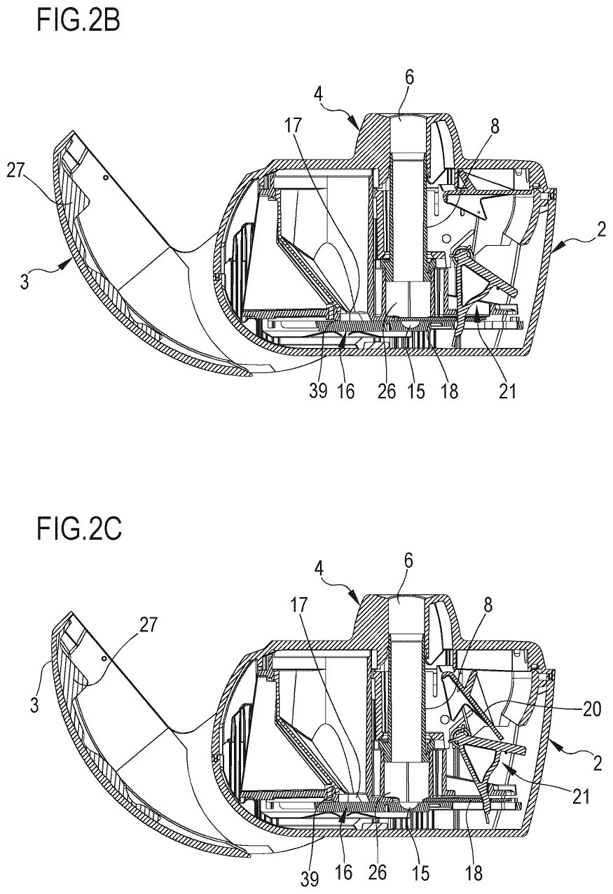

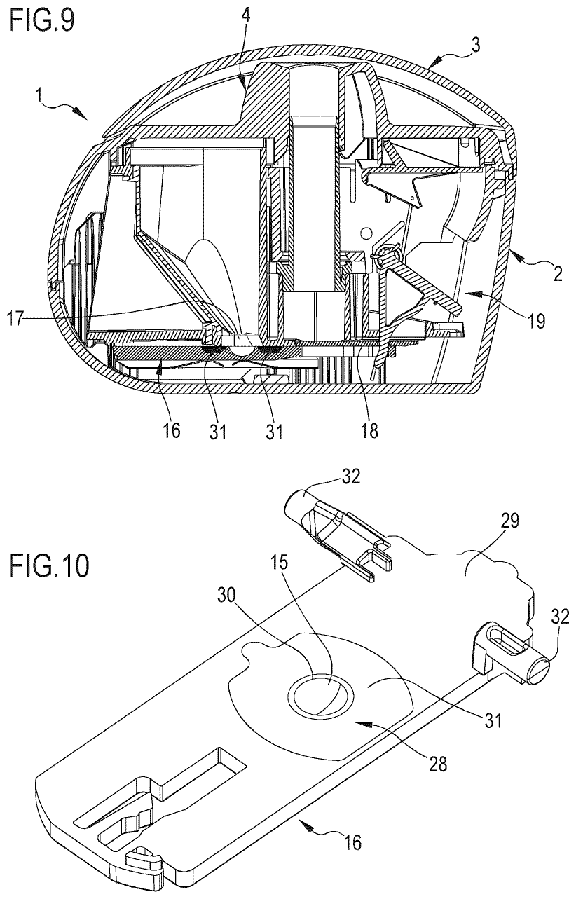

With reference to the appended drawings, FIGS. 1, 2A, 2B, 2C show a first embodiment, FIG. 9 shows a variant of the first embodiment and FIG. 14 shows a second embodiment of a powder inhaler 1 according to the present invention. The powder inhaler 1 of these non-limiting examples may be similar to the inhalers disclosed in document WO 2004/012801, WO 2016/000983, WO2021/105440 by the same Applicant.

The powder inhaler 1 comprises a casing 2 and a cover 3 being pivotably or rotatably coupled to the casing 2. As can be taken from FIG. 1, the cover 3 can be opened to reveal a mouthpiece 4 through which a user can inhale a powdered medicament. At an upper front side of the mouthpiece 4, air inlets 5 are formed in the casing 2.

The casing 2 is a closed shell made of thermoplastic material (e.g. ABS) and comprises lateral sides, an upper side and a lower side (upper and lower with respect to the orientation of the powder inhaler 1 of FIGS. 1, 2A-2C, 9 and 14). The mouthpiece 4 protrudes from the upper side and has an external shape of truncated cone tapering towards an opening 6 fashioned in a top portion (smaller base) of the mouthpiece 4.

The cover 3 is hinged to the casing 2 and can be rotated between a closed position, shown in FIGS. 2A, 9 and 14 in which the cover 3 encloses the mouthpiece 4, and an open position, shown in FIGS. 1, 2B and 2C, in which the cover 3 is spaced from the mouthpiece 4 to expose said mouthpiece 4 for use.

The powder inhaler 1 comprises a container 7 for storing a powdered medicament, an inhalation channel 8 connected to the opening 6 of the mouthpiece 4 and a dispensing device 9. The inhalation channel 8 has a first opening connected to the mouthpiece 4 and a second opening, opposite with respect to the first opening.

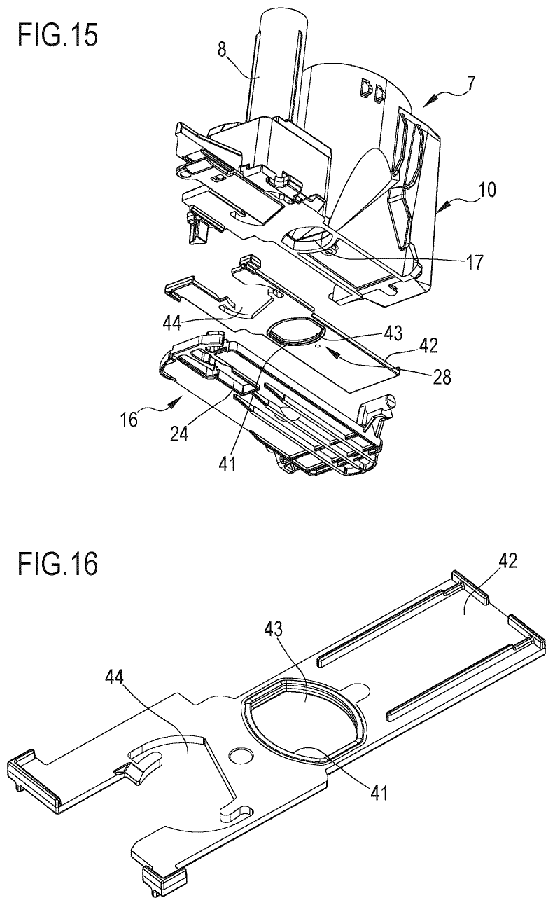

All these elements are part of sub-assembly 10, shown in FIG. 15, housed inside the casing 2.

As is shown in FIGS. 2A-2C, 9 and 14, the container 7 is a container with integral desiccant. The container 7 comprises a medicament chamber 11 storing the powdered medicament and a desiccant chamber 12 storing a desiccant for absorbing moisture that may have entered the medicament chamber 11.

The desiccant chamber 12 is separated from the medicament chamber 11 by a separate permeable membrane 13. This permeable membrane 13 is of a different permeability than the permeability between either the desiccant or the medicament to the outside environment. The permeability of the membrane 13 can be achieved, for example, by making it of a different material and/or a thinner section than the main body of the container 7. Foils may be used to seal both the medicament chamber 11 and the desiccant chamber 12.

The container 7, in particular the medicament chamber 11, is filled or is configured to be filled with an amount of powdered medicament corresponding to a plurality of doses, e.g. up to 100-200 doses. For instance, the powdered medicament is a pharmaceutical composition comprising a pharmaceutically acceptable salt of formoterol, e.g. in combination with a pharmaceutically acceptable salt of glycopyrronium and/or beclometasone dipropionate (BDP).

The desiccant is contained in a bag able of being inserted in the desiccant chamber 12 or the desiccant is in the form of a single tablet able of being inserted in the desiccant chamber 12. The desiccant is or comprises molecular sieves made of a material with pores of uniform size, for instance alkaline salts of aluminosilicates, called zeolites, or aluminophosphates or porous glass or active carbon or artificial zeolites. The molecular sieves are configured to absorb small molecules such as molecules of water.

The dispensing device 9 comprises a metering device 14 having a dosing recess 15. The metering device 14 shown in the attached Figures comprises a shuttle 16 shaped like a plate and provided with said dosing recess 15.

The dispensing device 9 is movable, with respect to the container 7 and with respect to the inhalation channel 8, between an idle state (FIGS. 2A, 9 and 14), in which a dosing recess 15 is in communication with an opening 17 of the container 7 so as to be filled with a dose of the powdered medicament, and a triggered state (FIG. 2C), in which the dosing recess 15 is in communication with the inhalation channel 8 for enabling inhalation of the dose of the powdered medicament contained in the dosing recess 15 through the mouthpiece 4.

The shuttle 16 is placed between the sub-assembly 10 and a bottom wall of the casing 2. The shuttle 16 is slidingly moveable between a filling position (FIGS. 2A, 9 and 14) and an inhalation position (FIGS. 2B and 2C). The filling position corresponds to the idle state of the metering device 14, in which the dosing recess 15 is in alignment with the opening 17 of the container 7 and faces said opening 17 so as to be filled with the dose of the powdered medicament. The inhalation position corresponds to an armed state (FIG. 2B) which will be detailed later and to the triggered state (FIG. 2C) of the metering device 14, in which the dosing recess 15 is in alignment with the inhalation channel 8.

The shuttle 16 is mechanically coupled to the cover 3 such that an opening of the cover 3 beyond a range of rotational movement from the closed position causes the shuttle 16 to move from the filling position to the inhalation position. Closing of the cover 3 causes the shuttle 16 to move back from the inhalation position to the filling position.

The metering device 14 further comprises a protective member 18 provided between the shuttle 16 and the inhalation channel 8. The protective member 18 is a plate arranged between the second opening of the inhalation channel 8 and the shuttle 16. The protective member 18 is parallel with respect to the shuttle 16 and is slidingly movable on or above the shuttle 16 between a closed position and an open position.

In the closed position, the protective member 18 is shifted backwards towards the second opening of the inhalation channel 8 and towards the container 7. In the closed position, a rear part of the protective member 18 may at least in part close the second opening of the inhalation channel 8. In the open position, the protective member 18 is shifted forward towards a wall of the casing 2. In the open position, a rear part of the protective member 18 leaves the second opening of the inhalation channel 8 open. The protective member 18 is in the closed position when the shuttle 16 is in the filling position (FIGS. 2A, 9 and 14). The protective member 18 may be moved between the closed position and the open position when the shuttle 16 is in the inhalation position (FIGS. 2B and 2C).

Therefore, the metering device 14 is configured to take the three different states cited above (idle, armed, triggered) and these states are determined by the positions of the shuttle 16 and of the protective member 18.

In the idle state (FIGS. 2A, 9 and 14), the shuttle 16 is in the filling position and the protective member 18 is in the closed position. The protective member 18 does not cover the dosing recess 15. The dosing recess 15 is communication with the opening of the container 7 to receive the medicament dose. In the armed state (FIG. 2B), the shuttle 16 is in the inhalation position and the protective member 18 is in the closed position. The protective member 18 covers the dosing recess 15. The protective member 18 prevents the powdered medicament contained in the dosing recess 15 from entering the inhalation channel 8 and being lost in case of rotation or movement of the powder inhaler 1 in oblique position before the inhalation manoeuvre or if the user blows into the mouthpiece 4. In the triggered state (FIG. 2C), the shuttle 16 is in the inhalation position and the protective member 18 is in the open position. The protective member 18 does not cover the dosing recess 15, thereby exposing the dosing recess 15 to the inhalation channel 8 so as to enable a user to inhale the dose of the powdered medicament contained in the dosing recess 15.

The dispensing device 9 further comprises a breath or inhalation actuated mechanism 19 coupled to the protective member 18. The inhalation actuated mechanism 19 comprises an inhalation actuated member 20 shaped like a flap, a coupling member 21 and a resilient element 22 (spring) arranged on the coupling member 21. The flap 20 is coupled to the protective member 18 through the coupling member 21 such that, if there is an inhalation suction force exceeding a predetermined value, the flap 20 is moved from a first position to a second position, thereby causing the protective member 18 to move from the closed position to the open position. The flap 20 is placed inside the casing 2 and close to the air inlets 5. In the first position (FIGS. 2A, 9 and 14), the flap 20 separates the air inlets 5 from the inhalation channel 8 and seats in a main airflow path. The flap 20 provides a resistance if the user blows into the device giving positive feedback. In the second position, (FIG. 2C) the flap 20 is rotated with respect to the first position to open the air inlets 5 and to allow air flowing through the air inlets 5 into the inhalation channel 8 and out of the mouthpiece 4. The resilient element 22 is arranged such that said resilient element 22 holds the flap 20 in its first position. When the shuttle 16 is pushed forward by opening the cover 3, the resilient element 22 is compressed and charged and the reset force exerted on the flap 20 is released, so that the flap 20 can pivot or rotate from the first position into the second position that is pivoted downward relative to the first position if there is a sufficient high inhalation suction force in the inhalation channel 8.

The flap 20 is hinged to the casing 2 in order to rotate between the first position and the second position around a respective rotation axis which is substantially perpendicular to a main axis Z-Z of the inhalation channel 8. The coupling member 21 is also hinged to the casing 2 in order to rotate between a respective first position and second position around a respective rotation axis Y-Y which is substantially perpendicular to the main axis Z-Z of the inhalation channel 8.

The coupling member 21 comprises an arm, not shown, protruding towards the flap 20 and engaged with the flap 20 such that the clockwise rotation of the flap 20 from the first position to the second position causes a counterclockwise rotation of the coupling member 21 from its respective first position towards its respective second position.

The coupling member 21 comprises a prolongation 23 engaging with an opening formed in the protective member 18 in order to move the protective member 18 from the closed position to the open position when the coupling member 21 moves from its respective first position to its respective second position and vice-versa.

The prolongation 23 of the coupling member 21 is also moveably arranged in a longitudinal opening 24 which is formed in the shuttle 16 along its longitudinal direction, such that said prolongation 23 can freely move in the longitudinal opening 24, while a movement of the shuttle 16 from the inhalation position to the filling position causes the prolongation 23 of the coupling member 21 to abut against an edge of the longitudinal opening 24 thereby moving the coupling member 21 back into its initial first position.

The inhaler disclosed in the attached Figures further comprises a de-agglomerator arrangement 25 that is coupled to the second end of the inhalation channel 8 opposite the mouthpiece 4. The de-agglomerator arrangement 25 delimits a vortex chamber 26 and is constructed such that it generates a cyclonic airflow resulting in a strong velocity gradient. The protective member 18 is slidable on the shuttle 16 between its closed position, in which is covers the dosing recess 15, and its open position, in which it exposes the dosing recess 15 to the de-agglomerator arrangement 25 and the inhalation channel 8 when the metering member 14 is in the inhalation position, so that the dose of the powdered medicament can be inhaled through the de-agglomerator arrangement 25 and the inhalation channel 8 as well as the mouthpiece 4.

The internal mechanisms and functioning of the powder inhaler 1 disclosed here above are similar to those disclosed in documents WO 2004/012801, WO 2016/000983, WO2021/105440 by the same Applicant.

The powder inhaler 1 shown in the attached Figures may also comprise a sealing element 27 housed in the cover 3 and made of a material (e.g. silicone or a thermoplastic elastomer (TPE)) that is more deformable than a material of the mouthpiece 4, so that, when the cover 3 is engaged with the casing 2 and closes the mouthpiece 4 (FIGS. 2A, 9 and 14), a first portion of the sealing element 27 is coupled to the opening 6 of the mouthpiece 4 to tight close said opening 6 of the mouthpiece 4 and a second portion of the sealing element 27 is coupled to the air inlets 5 to tight close said air inlets 5.

The powder inhaler 1 may also comprise a dose counting unit, not shown in the embodiment of the attached drawings, contained into the casing 2 and coupled both to the inhalation actuated mechanism 19 and to the closure of the cover 3 after an efficacious inhalation has occurred. The casing 2 may also comprise a window or an opening for displaying the number of doses taken or the number of doses left in the container 7, this number being counted by the dose counting unit. For instance, the dose counting unit is the same or similar to the one disclosed in document WO 2004/012801.

According to the present invention, the powder inhaler 1 further comprises a sealing device 28 operationally active at a coupling zone of the shuttle 16 with the container 7 when the shuttle 16 is in the filling position, said coupling zone circumscribing the dosing recess 15 and the opening 17 of the container 7 and being interposed between the shuttle 16 and the container 7. The sealing device 28 removes or reduces gaps between the shuttle 16 and the container 7 and allows to prevent or at least reduce entry of moisture in the container 7 when the shuttle 16 is in the filling position.

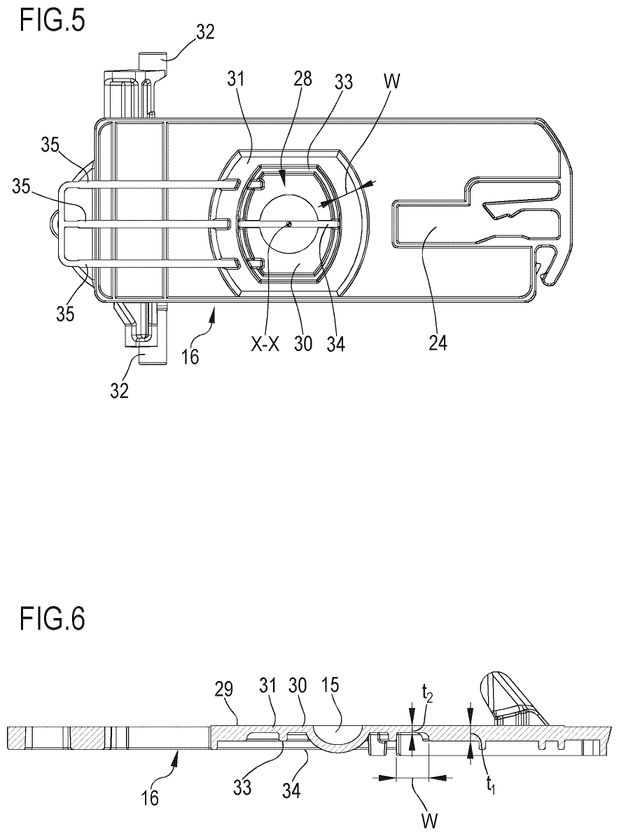

FIGS. 2A to 6 show a first illustrative embodiment of the sealing device 28 according to the invention.

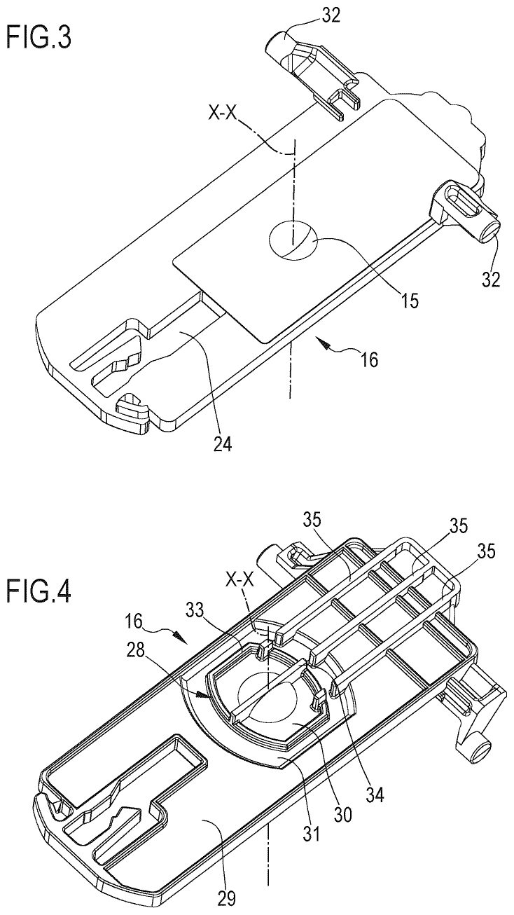

As can be seen in FIGS. 3, 4, 5 and 6, the shuttle 16 is shaped like a plate made of a single piece of plastic, e.g. acrylonitrile butadiene styrene copolymer (ABS). The shuttle 16 comprises a main part 29, a dosing part 30 and a deformable portion 31 surrounding the dosing recess 15 and connecting the dosing part 30 to the main part 29. The cited sealing device 28 is defined or comprises said deformable portion 31.

The dosing part 30 comprises the dosing recess 15 which is fashioned in an upper face of the dosing part 30 and is shaped like a cup having a circular edge, i.e. is a spherical cap recess.

The main part 29 surrounds the dosing part 30 and is coupled to the casing 2 and/or to the sub-assembly 10 in order to shift between the filling position and the inhalation position. To this aim, the shuttle 16 comprises pins 32 protruding laterally from the main part 29 and accommodated in tracks of the casing 2 and/or to the sub-assembly 10, so as to be able to slide. The longitudinal opening 24 is also fashioned in the main part 29.

The deformable portion 31 is a flat portion shaped like a band surrounding the dosing recess 15 and the dosing part 30. The deformable portion 31 and the dosing part 30 have a second wall thickness t2 which is smaller than a first wall thickness t1 of the main part 29. For instance, the first wall thickness t1 is 1.1 mm and the second wall thickness t2 is 0.45 mm. Therefore, a ratio t2/t1 of the second wall thickness t2 to the first wall thickness t1 is about 0.4.

The deformable portion 31 extends radially with respect to a central axis X-X of the dosing recess 15 perpendicular to the shuttle 16 and has an average width wₐᵥ measured along radial directions, wherein the average width wₐᵥ is an average of a radial width w measured on the 360°. For instance, the average width wₐᵥ is about 1.9 mm and a ratio t2/wₐᵥ of the second wall thickness t2 to the average width wₐᵥ is 0.24.

The dosing part 30 is delimited by a peripheral stiffening rib 33 which is fashioned on a side of the shuttle 16 opposite the dosing recess 15 and is surrounded by the deformable portion 31. A further straight stiffening rib 34 is placed on the side of the dosing part 30 opposite the dosing recess 15, is aligned to a direction of movement of the shuttle 16, passes through the central axis X-X of the dosing recess 15 and connects to the peripheral stiffening rib 33. This straight stiffening rib 34 does not extend over the deformable portion 31 and over the main part 29. The shuttle 16 comprises further stiffening ribs 35 on the main part 29 which are not connected to the dosing part 30.

Therefore, thanks to the different wall thickness with respect to the main part 29 and to the stiffening ribs 33, 34, 35 on the dosing part 30 and main part 29, the deformable portion 31 is less stiff than the main part 29 and the dosing part 30 along a direction perpendicular to a plane in which the shuttle 16 lies, i.e. the plane perpendicular to the central axis X-X of the dosing recess 15.

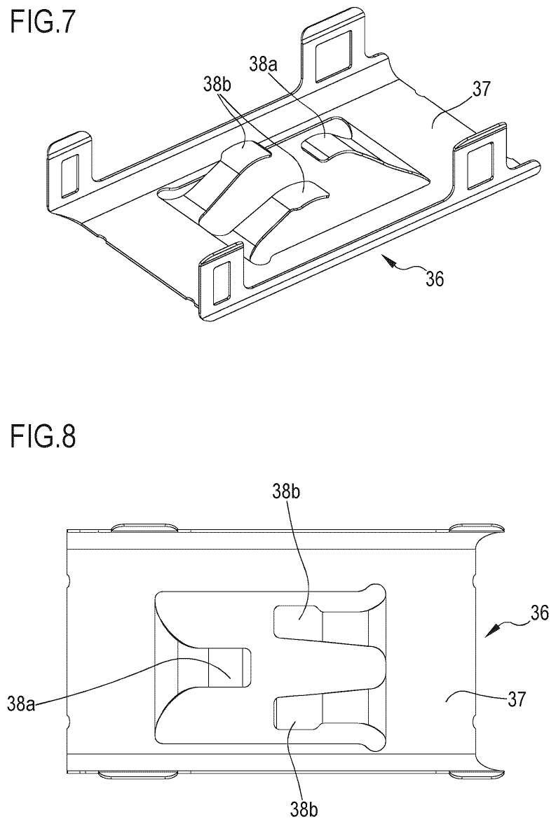

As shown in FIG. 2A, a spring 36 is interposed between a bottom wall of the casing 2 and the dosing part 30 and is configured to push said dosing part 30 against the opening 17 of the container 7 by deforming the deformable portion 31 when the shuttle 16 is in the filling position. The spring 36 comprises a base 37 anchored to the casing 2 and/or to the sub-assembly 10 and three elastic arms 38a, 38b protruding from the base 37 towards the shuttle 16 (FIGS. 7 and 8). The shuttle 16 slides with respect to the spring 36 when moving between the filling position and the inhalation position.

When the shuttle 16 is in the filling position, a central arm 38a of said elastic arms 38a, 38b pushes against the straight stiffening rib 34 of the dosing part 30 at a center of the dosing recess 15 and two lateral arms 38b of said elastic arms 38a, 38b push against the peripheral stiffening rib 33, in order to press a zone of the dosing part 30 surrounding the dosing recess 15 against an edge 39 of the opening 17 of the container 7 (FIG. 2A) by deforming the deformable portion 31.

FIGS. 9 to 13 show a variant of the first embodiment which differs from the first embodiment due to a different structure of the shuttle 16.

The deformable portion 31 of the shuttle 16 according to this variant comprises an elastomeric material, e.g. silicone or a thermoplastic elastomer (TPE) for medical-pharmaceutical applications, like styrene block copolymers (TPS (TPE-s)), thermoplastic polyolefin elastomers (TPO (TPE-o)), thermoplastic vulcanizates (TPV (TPE-v or TPV)), thermoplastic polyurethanes (TPU), thermoplastic copolyesters (TPC (TPE-E)), thermoplastic polyamides (TPA (TPE-A)), not classified thermoplastic elastomers (TPZ).

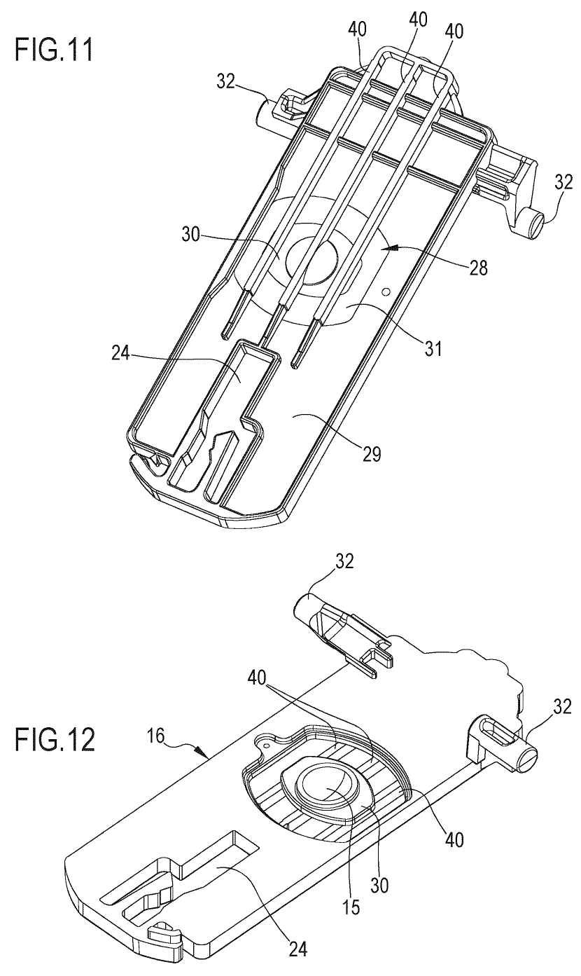

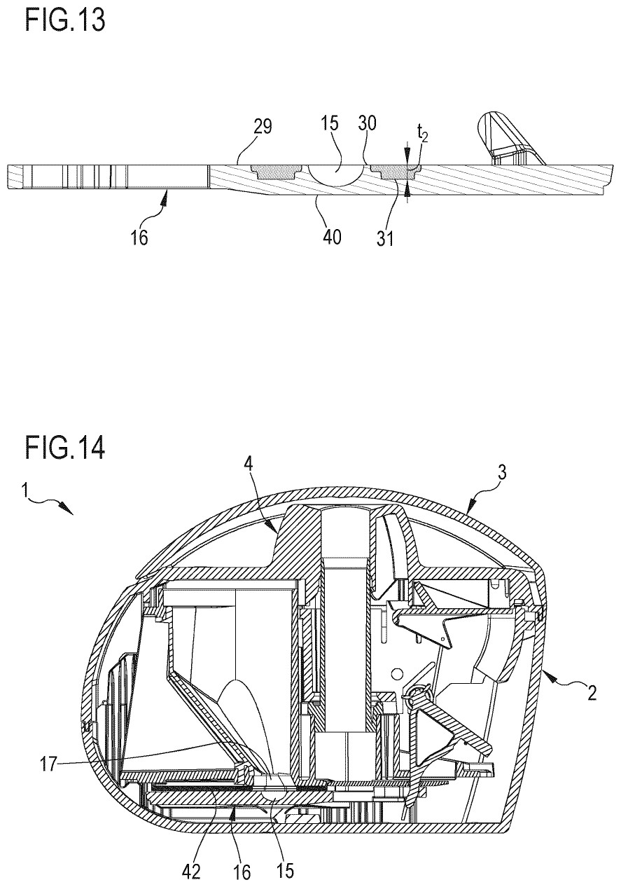

As better seen in FIGS. 11 and 12, the main part 29 and the dosing part 30 are spaced apart one from the other and connected to each other through connecting ribs 40 joining said main part 29 to the dosing part 30. The connecting ribs 40 are aligned to a direction of movement of the shuttle 16 between the filling position and the inhalation position and are positioned on a side of the shuttle 16 opposite the dosing recess 15. The main part 29, the dosing part 30 and the connecting ribs 40 are made of single piece of plastic, e.g. acrylonitrile butadiene styrene (ABS).

The deformable portion 31 of elastomeric material is co-molded or over-molded to the main part 29 and to the dosing part 30 and fills an annular gap between said main part 29 and dosing part 30. The main part 29 and the dosing part 30 have steps to accommodate the elastomeric material such that the deformable portion 31 is flush to an upper surface of the main part 29 and of the dosing part 30.

Also the deformable portion 31 of elastomeric material is a flat portion shaped like a band surrounding the dosing recess 15 and the dosing part 30. The deformable portion 31 and the dosing part 30 have a second wall thickness t2 which may be equal to (not shown in FIG. 13) or smaller than the first wall thickness t1 of the main part 29.

For instance, the first wall thickness t1 and the second wall thickness t2 are the same, e.g. equal to 1.1 mm.

The deformable portion 31 extends radially with respect to a central axis X-X of the dosing recess 15 perpendicular to the shuttle 16 and has an average width wₐᵥ measured along radial directions, wherein the average width wₐᵥ is an average of a radial width w measured on the 360°. For instance, the average width wₐᵥ of the deformable portion 31 of elastomeric material is about 1.9 mm and a ratio t2/wₐᵥ Of the second wall thickness t2 to the average width wₐᵥ is about 0.6.

FIGS. 14 to 17 show a second embodiment of the inhaler 1 in which the sealing device 28 is not integrated in the shuttle 16 but, differently, is a gasket 41 placed at the coupling zone which circumscribes the dosing recess 15 and the opening 17 of the container 7 and is interposed between the shuttle 16 and the container 7.

In this embodiment, the inhaler 1 comprises a support plate 42 made of a plastic material such as polycarbonate (PC), polypropylene (PP), acrylonitrile-butadiene-styrene copolymer (ABS) or cyclic olefin copolymer (COC). The support plate 42 is sandwiched between the container 7 and the shuttle 16. The support plate 42 is anchored to the casing 2 and/or to the sub-assembly 10 so that it is stationary with respect to these elements. The support plate 42 has a through opening 43 and a through inhalation passage 44.

The through opening 43 faces the opening 17 of the container 7. The gasket 41 is made of an elastomeric material, e.g. a thermoplastic elastomer (TPE) or silicone, and is co-molded with or over-molded to an edge of the through opening 43.

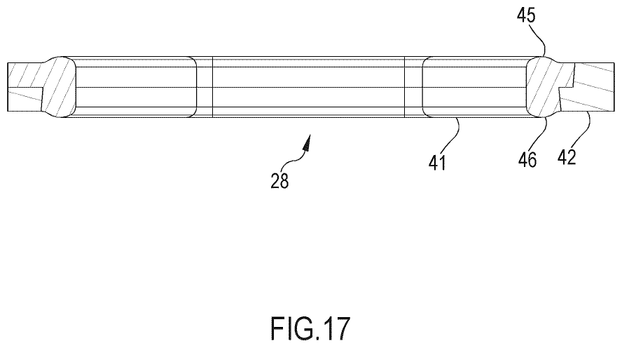

The cross section of FIG. 17 shows that the gasket 41 has a raised bead 45 protruding towards the container 7 and a raised bead 46 protruding towards the shuttle 16.

The shuttle 16 may be the same or similar to the one disclosed in WO 2004/012801, WO 2016/000983 or WO2021/105440. The arms 38a, 38b of the spring 36 pushes the shuttle 16 against the support plate 42 and pushes the support plate 42 against the sub-assembly 10, so that the gasket 41 is sandwiched between the shuttle 16 and the casing 2 and seals the opening 17.

### Example

The different embodiments of the invention were tested.

Test conditions: 30° C. and 75% RH.

The desiccant was removed from the desiccant chamber 12 and the medicament chamber 11 was filled with 3 g of calcium chloride anhydrous to evaluate the moisture ingress in said medicament chamber 11.

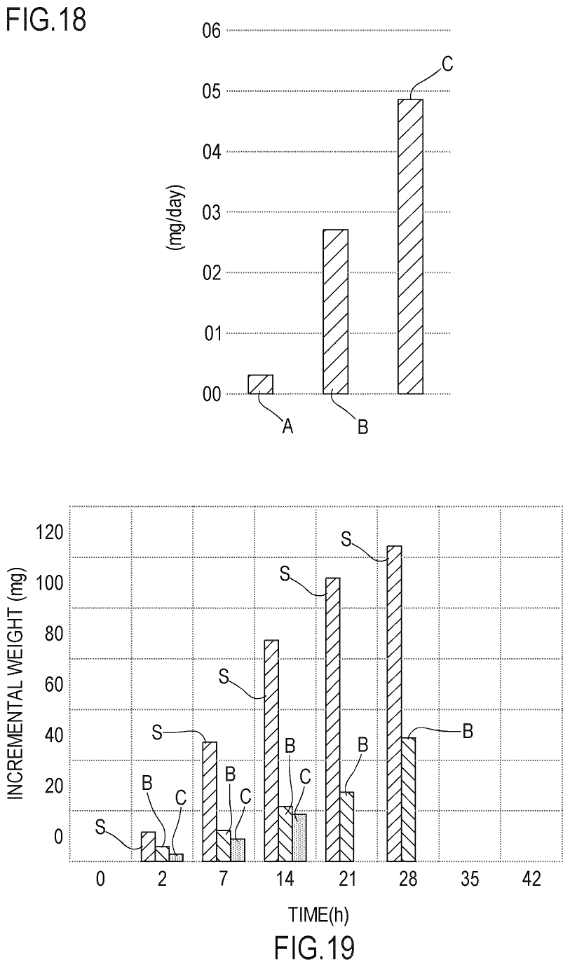

FIG. 18 shows a differential weight per day (mg/day) due to moisture absorption of the different embodiments/variants of the invention with respect to a standard powder inhaler, wherein:
A—first embodiment of FIGS. 2A to 6;
B—variant of FIGS. 9 to 13;
C—second embodiment of FIGS. 14 to 17.

FIG. 19 is a chart showing a cumulative/incremental weight (mg) over time (h) due to moisture absorption in a standard powder inhaler and in the embodiments/variants of the invention, wherein:
S—powder inhaler with standard shuttle as disclosed in WO2004/012801 or WO 2016/000983;
B—variant of FIGS. 9 to 13;
C—second embodiment of FIGS. 14 to 17.

As shown in FIG. 18, all the embodiments/variants A, B and C exhibited an improvement in the humidity protection with respect to the standard powder inhaler.

As shown in FIG. 19, after 14 days, both the embodiments/variants B and C showed a saving of about 70 mg of water and after 28 days the variant B show a saving of about 75 mg of water.

### LIST OF PARTS

powder inhaler 1
casing 2
cover 3
mouthpiece 4
air inlets 5
opening 6 of the mouthpiece
container 7
inhalation channel 8
dispensing device 9
sub-assembly 10
medicament chamber 11
desiccant chamber 12
permeable membrane 13
metering device 14
dosing recess 15
shuttle 16
opening 17 of the container
protective member 18
breath or inhalation actuated mechanism 19
inhalation actuated member or flap 20
coupling member 21
resilient element 22
prolongation 23
longitudinal opening 24
de-agglomerator arrangement 25
vortex chamber 26
a sealing element 27
sealing device 28
main part 29
dosing part 30
deformable portion 31
pins 32
peripheral stiffening rib 33
stiffening rib 34
further stiffening ribs 35
spring 36
base 37
elastic arms 38a, 38b
edge 39 of the opening of the container
connecting ribs 40
gasket 41
support plate 42
through opening 43
through inhalation passage 44
raised bead 45
raised bead 46
central axis X-X

1.-15. (canceled)

16. A powder inhaler, comprising:
a casing having a mouthpiece;
an inhalation channel housed in the casing and connected to the mouthpiece;
a container housed in the casing for storing a powdered medicament;
a metering device comprising a shuttle having a dosing recess, wherein the shuttle is movable between a filling position, in which the dosing recess is in alignment with an opening of the container and faces said opening so as to be filled with a dose of the powdered medicament, and an inhalation position, in which the dosing recess is in alignment with the inhalation channel, for enabling inhalation of the dose of the powdered medicament contained in the dosing recess through the mouthpiece; and
a sealing device operationally active at a coupling zone of the shuttle with the container when the shuttle is in the filling position, said coupling zone circumscribing the dosing recess and the opening.

17. The powder inhaler of claim 16, wherein
the shuttle comprises a main part and a dosing part;
the dosing part comprises the dosing recess;
the sealing device comprises a deformable portion surrounding the dosing recess and connecting the dosing part to the main part;
the deformable portion is less stiff than the main part along a direction perpendicular to a lying plane of the shuttle; and
at least one spring is interposed between the casing and the dosing part and is configured to push said dosing part against the opening of the container by deforming the deformable portion when the shuttle is in the filling position.

18. The powder inhaler of claim 17, wherein
the shuttle is shaped as a plate and the main part has a first wall thickness, and
the deformable portion has a second wall thickness equal to or smaller than the first wall thickness.

19. The powder inhaler of claim 18, wherein the dosing part comprises a peripheral stiffening rib surrounded by the deformable portion.

20. The powder inhaler of claim 18, wherein
the deformable portion has an average width, and
a ratio t2/wₐᵥ of the second wall thickness (t2) to the average width (wₐᵥ) is smaller than 0.4.

21. The powder inhaler of claim 17, wherein the main part, the deformable portion and the dosing part are made in a single piece.

22. The powder inhaler of claim 17, wherein the deformable portion is made of or comprises an elastomeric material.

23. The powder inhaler of claim 17, wherein the dosing part comprises at least one stiffening rib on a side of the shuttle opposite the dosing recess.

24. The powder inhaler of claim 17, wherein the shuttle comprises at least one connecting rib joining the main part to the dosing part.

25. The powder inhaler of claim 17, wherein the at least one spring contacts the dosing part at least at a center of the dosing recess.

26. The powder inhaler of claim 16, wherein the sealing device comprises a gasket placed at the coupling zone, at least when the shuttle is in the filling position.

27. The powder inhaler of claim 26, comprising a support plate sandwiched between the container and the shuttle and having a through opening facing the opening of the container, wherein the gasket is placed around said through opening.

28. The powder inhaler of claim 27, wherein the gasket is made of an elastomeric material and the support plate is made of plastic.

29. The powder inhaler of claim 26, wherein the gasket has a raised bead protruding towards the container and/or a raised bead protruding towards the shuttle.

30. The powder inhaler of claim 16, comprising a desiccant chamber placed inside the casing and containing or configured to contain a desiccant, wherein the desiccant comprises molecular sieves.
